(19) United States
(12) Patent Application Publication
(10) Pub. No.: US 2023/0310164 A1
(43) Pub. Date: Oct. 5, 2023

(54) KNEE PROSTHESIS WITH INCREASED PATELLA FREEDOM OF MOVEMENT

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Nathaniel M. Lenz, Germantown, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/020,455

(22) PCT Filed: Sep. 21, 2021
(86) PCT No.: PCT/US2021/051279

§ 371 Date: Feb. 9, 2023

Related U.S. Application Data
(60) Provisional application No. 63/082,316, filed on Sep. 23, 2020.

(51) Int. Cl. A61F 2/38

(52) U.S. Cl. CPC A61F 2/3877; A61F 2/3859

(57) ABSTRACT

Knee implants and components thereof are disclosed. A femoral component of a knee implant may include a reduced constraint region arranged on a portion of a patella path. The reduced constraint region may be configured to provide increased freedom-of-movement of a patella traveling over the reduced constraint region. The increased freedom-of-movement may be with respect to medial-lateral movement and/or internal-external rotation of the patella. The patella path may include a constraint region (e.g., a trochlear groove) positioned inferiorly of the reduced constraint region. A patella may travel within the reduced constraint region during extension (or partial flexion) and within the trochlear groove during flexion. A prosthetic patella having a raised portion is also disclosed. The raised portion may be configured to facilitate engagement of the prosthetic patella with the trochlear groove of the femoral component as the patella transitions from the reduced constraint region to the trochlear groove.

### CROSS-REFERENCE TO RELATED APPLICATIONS

This is a non-provisional of, and claims the benefit of the filing date of, pending U.S. Provisional Pat. Application No. 63/082,316, filed Sep. 23, 2020, entitled “Knee Prosthesis with Increased Patella Freedom of Movement,” the entirety of which application is incorporated by reference herein.

### FIELD OF THE DISCLOSURE

The present disclosure generally relates to a knee prosthesis and, more particularly, to a femoral component and/or a patella implant of a knee prosthesis that are configured to facilitate increased freedom of movement of a patella or a patella implant during articulation of the femoral component.

### BACKGROUND OF THE DISCLOSURE

Significant disease or injury affecting the knee joint may be treated by a total knee arthroplasty (TKA) that surgically replaces the ends of the patient’s femur and tibia with a prosthetic knee device that may include a prosthetic femoral implant or component, a tibial implant or component, and, in some cases, a patella implant or component (terms prosthesis, implant, component, and device are used interchangeably herein without the intent to distinguish or limit). The femoral component may be placed on a patient’s distal femur after appropriate resection of the patient’s femur. The tibial component may include a tibial tray that generally conforms to the patient’s resected proximal tibia. The tibial component may also include a stem that extends from the tray in order to extend into a surgically formed opening in the patient’s intramedullary canal of the tibia. A plastic or polymeric insert or bearing may be arranged between the tray of the tibial component and the femoral component to provide a surface against which the femoral component may articulate as the patient’s knee moves between an extension position and a flexion position. The TKA procedure may also include replacement of the patient’s patella with a prosthetic patella, for example, to enhance the functioning and cooperation of the patellofemoral joint of the prosthetic knee device.

A common complaint of TKA patients is that the replaced knee does not function or feel like a normal knee. In more serious cases, patients complain of pain or discomfort during articulation of the prosthetic knee device. Anterior knee pain and instability are common factors in patient dissatisfaction with the TKA procedure. Conventional prosthetic knee devices produce kinematics different than the normal knee during gait, due to the complex nature of the knee joint and the motion of the femur, tibia, and patella relative to one another during flexion and extension. For example, patient pain and discomfort in the patellofemoral joint may arise due to constraints on movement of components, such as the patella, that may cause conflict between native anatomy and implanted components.

Thus, it would be beneficial to provide knee prosthetic devices that support natural operation of knee components, including facilitating component range of motion, to alleviate the pain and instability associated with conventional prosthetic knee devices.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure provides a femoral component of a prosthetic knee implant comprising a reduced constraint region arranged on a portion of a patella path. The reduced constraint region may be configured to provide increased freedom-of-movement of a patella traveling over the reduced constraint region. In some embodiments, the increased freedom-of-movement may be with respect to medial-lateral movement and/or internal-external rotation of the patella. The patella path may include a constraint region in the form of a trochlear groove inferior to the reduced constraint region. In some embodiments, a patella may travel within the reduced constraint region during extension (or partial flexion) and within the trochlear groove during flexion.

In some embodiments, the reduced constraint region may include a flattened proximal anterior flange region of the femoral component. In various embodiments, the reduced constraint region may have a length of about 1 percent to about 20 percent of the distal anterior flange region. In various embodiments, the reduced constraint region may have a length of 1 mm to about 20 mm.

In various embodiments, the reduced constraint region may be arranged in a region of the patella path where the patella travels during extension and/or partial states of flexion below a constrained flexion degree. In some embodiments, the reduced constraint region may be arranged in a region of the patella path where the patella travels during about 0 degrees flexion (i.e., extension or full extension). In some embodiments, the constrained flexion degree may be less than about 10 degrees flexion. In some embodiments, the constrained flexion degree may be less than 30 degrees flexion.

In some embodiments, the length of the reduced constraint region may be or may be determined to provide that patella is within the reduced constraint region during a threshold percentage of flexion. In some embodiments, the threshold percentage of flexion may be about 0 degrees to about 45 degrees.

In various embodiments, a patella may have increased medial-lateral movement within the patella path in the reduced constraint region compared with within a trochlear groove of the patella path. In some embodiments, percentage medial-lateral movement increase may be about 105 percent to about 150 percent. In some embodiments, percentage medial-lateral movement increase may be about 1 mm to about 20 mm. In various embodiments, the medial-lateral movement increase may be achieved at extension (or substantially near extension, such as less than about 5 percent flexion).

In various embodiments, a patella may have increased internal-external rotation within the patella path in the reduced constraint region compared with within a trochlear groove of patella path. In some embodiments, the percentage internal-external rotation increase may be about 105 percent to about 150 percent. In various embodiments, the internal-external rotation increase may be achieved at extension (or substantially near extension, such as less than about 5 percent flexion).

In some embodiments, the reduced constraint region may be characterized by a maximum sulcus angle of about 150 degrees to about 180 degrees. In various embodiments, the reduced constraint region may be characterized by an average sulcus angle of about 150 degrees to about 180 degrees. In exemplary embodiments, the reduced constraint region may be characterized by a maximum groove depth of about 1 mm to about 2 mm. In exemplary embodiments, the reduced constraint region may be characterized by an average groove depth of about 1 mm to about 2 mm.

The present disclosure provides a prosthetic patella of a prosthetic knee implant having a raised portion. In some embodiments, the raised portion is arranged on a distal region of the posterior side of prosthetic patella. In various embodiments, the raised portion may be configured to facilitate engagement of the prosthetic patella with a trochlear groove of a femoral component as the prosthetic patella transitions from a reduced constraint region of the femoral component to the trochlear groove.

In some embodiments, the raised portion may be formed to flair out moving from the outer edge to the center of posterior side. In various embodiments, the raised portion may have a height of about 1 mm to about 5 mm. In some embodiments, the location of the raised portion may be determined based on various factors, such as whether patella tracks medial, lateral, or central in extension

Some embodiments may provide a knee implant system having a femoral component and a prosthetic patella. The femoral component may comprise a reduced constraint region arranged on a portion of a patella path. The reduced constraint region may be configured to provide increased freedom-of-movement of a patella traveling over the reduced constraint region. In some embodiments, the increased freedom-of-movement may be with respect to medial-lateral movement and/or internal-external rotation of the patella. The patella path may include a constraint region in the form of a trochlear groove inferior to the reduced constraint region. In some embodiments, a patella may travel within the reduced constraint region during extension (or partial flexion) and within the trochlear groove during flexion.

In various embodiments, the patella may have a raised portion. In some embodiments, the raised portion is arranged on a distal region of the posterior side of prosthetic patella. In various embodiments, the raised portion may be configured to facilitate engagement of the prosthetic patella with a trochlear groove of a femoral component as the prosthetic patella transitions from a reduced constraint region of the femoral component to the trochlear groove.

Some embodiments may provide a surgical method of implanting a prosthetic knee system having a femoral component, a tibial component, and a patella in a patient. The method may include the step of affixing the prosthetic knee system to the bony anatomy of the patient.

In various embodiments, the femoral component may comprise a reduced constraint region arranged on a portion of a patella path. The reduced constraint region may be configured to provide increased freedom-of-movement of a patella traveling over the reduced constraint region. In some embodiments, the increased freedom-of-movement may be with respect to medial-lateral movement and/or internal-external rotation of the patella. The patella path may include a constraint region in the form of a trochlear groove inferior to the reduced constraint region. In some embodiments, a patella may travel within the reduced constraint region during extension (or partial flexion) and within the trochlear groove during flexion.

In various embodiments, the prosthetic patella may include a raised portion. In some embodiments, the raised portion is arranged on a distal region of the posterior side of prosthetic patella. In various embodiments, the raised portion may be configured to facilitate engagement of the prosthetic patella with a trochlear groove of a femoral component as the prosthetic patella transitions from a reduced constraint region of the femoral component to the trochlear groove

Embodiments of the present disclosure provide numerous advantages. In one non-limiting example advantage, a knee implant system may include a femoral component having a reduced constraint region configured to provide increased freedom-of-movement to a patella, for example, when the knee is in extension and there is more variability in native patella position while transitioning the patella into the conforming trochlea for load transfer between the patella and the femur when the knee flexes. Accordingly, knee implants according to some embodiments may provide for more natural patella movement compared with conventional systems. In addition, knee implants according to some embodiments may operate to reduce (or even eliminate) residual pain or discomfort in the anterior knee due to the kinematics of the femoropatellar joint after a TKA.

Further features and advantages of at least some of the embodiments of the present disclosure, as well as the structure and operation of various embodiments of the present disclosure, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 shows a perspective view of an embodiment of a conventional left knee prosthesis;

FIG. 2 shows various side views of a knee prosthesis (e.g., femoral component and a patella) showing the kinematics of the knee prosthesis during extension and varying degrees of flexion;

FIG. 3 shows a side view of an embodiment of a femoral component in accordance with the present disclosure, the femoral component may be used in connection with the knee prosthesis shown in FIGS. 1 and 2;

FIG. 4A shows a front view of an embodiment of a femoral component in accordance with the present disclosure, the femoral component may be used in connection with the knee prosthesis shown in FIGS. 1 and 2;

FIG. 4B shows a side view of an alternate embodiment of a femoral component in accordance with the present disclosure, the femoral component may be used in connection with the knee prosthesis shown in FIGS. 1 and 2;

FIG. 4C shows a front view of an alternate embodiment of a femoral component in accordance with the present disclosure, the femoral component may be used in connection with the knee prosthesis shown in FIGS. 1 and 2;

FIG. 5A shows a side view and corresponding sectional views of an embodiment of a femoral component in accordance with the present disclosure, the femoral component may be used in connection with the knee prosthesis shown in FIGS. 1 and 2;

FIGS. 5B and 5C show patella track information for an illustrative sectional view of FIG. 5A in accordance with the present disclosure;

FIG. 6 shows a side view and corresponding sectional views of an embodiment of a femoral component in accordance with the present disclosure, the femoral component may be used in connection with the knee prosthesis shown in FIGS. 1 and 2;

FIG. 7 shows a perspective view of an embodiment of a patella implant in accordance with the present disclosure; and

FIG. 8 shows a graph of patella medial-lateral tracking during degrees of knee flexion for simulated knee prosthesis in accordance with the present disclosure.

The drawings are not necessarily to scale. The drawings are merely representations, not intended to portray specific parameters of the disclosure. The drawings are intended to depict example embodiments of the disclosure, and therefore are not to be considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

### DETAILED DESCRIPTION

Embodiments of an improved knee prosthesis will now be described more fully hereinafter with reference to the accompanying drawings, in which preferred embodiments of the present disclosure are presented. As will be described and illustrated, in some embodiments, the improved knee prosthesis may include a femoral component for use in a knee prosthesis, the femoral component having an articular surface configured to provide a patella path for engaging and guiding a patella or a patella implant during extension and flexion of the knee. In some embodiments, the patella path may include a reduced patella constraint (or reduced constraint) portion, region, segment, or area (terms used interchangeably herein without the intent to distinguish) configured to allow for increased freedom-of-movement of the patella or a patella implant as it travels over this portion of the patella path in comparison to the range of movement provided by a patella path of a conventional femoral component. For example, the reduced constraint region may facilitate greater freedom-of-movement of the patella or a patella implant compared with movement of the patella or a patella implant during travel within a trochlear groove or patella track of a conventional prosthetic femoral component. The increased freedom-of-movement may be with respect to one or more movements of the patella or a patella implant, including, without limitations, medial-lateral translational movement, internal-external rotational movement (e.g., patella tilt), and/or the like.

As described in more detail in the present disclosure, in some embodiments, the reduced constraint region may be configured as a flattened region on a proximal anterior flange of the femoral component. In various embodiments, the reduced constraint region may be arranged in a region of the patella path where the patella travels during extension and/or partial states of flexion.

In various embodiments, the patella path may include the reduced constraint region and a patella constraint (or constraint) region. In various embodiments, the constraint region may be or may include a trochlear groove or patella track. In some embodiments, the femoral component may include a constraint region (i.e., trochlear groove) that extends less superior from the distal femoral condyles in comparison to trochlear grooves of conventional femoral components to allow more freedom-of-movement of the patella or patella implant in extension and/or partial states of flexion.

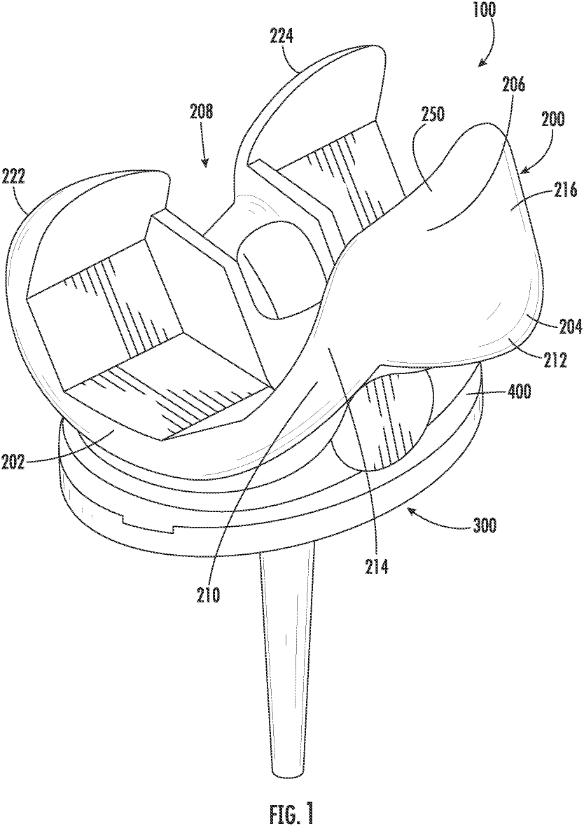

Referring to FIG. 1, existing femoral components include an anterior flange sized and shaped to cover as much of the resected anterior bone as possible, with a trochlear groove for the patella or patella implant extending to the superior edge of the anterior flange. Multiple designs exist for forming a trochlear groove (for instance, straight, angled, offset, S-curve, funnel, and/or the like) and a prosthetic patella (for example, round dome/sombrero, centralized oval, offset oval, anatomic, mobile bearing, and/or the like). Different methods have been used to position the femur (for instance, align internal-external rotation based on anterior-posterior axis/Whiteside’s line, epicondyles, posterior condyles, and/or the like) and patella (medialized, best coverage, lateral facetectomy to remove uncovered bone with medialized implant, non-resurfaced).

The most common complications after total knee arthroplasty (TKA) are related to femoropatellar problems, with residual pain in the anterior knee manifested in a significant population of patients. Altered patellar kinematics has been cited as a main contributor to the development of patellar complications after TKA. For example, in conventional femoral implants, the trochlear groove or patella track extends further superior than the trochlear groove in the native bone. Accordingly, movement of the patella or patella implant with a conventional femoral implant, particularly in extension, may be constrained in a portion of the patella path where the patella or patella implant is not as restricted in native anatomy. A femoral component of a knee implant having a reduced constraint region of a patella path may allow for freedom-of-movement of the patella or patella implant that corresponds or more closely corresponds to movement of the patella in native anatomy.

Accordingly, some embodiments may provide multiple technological advantages over existing knee implants. One non-limiting example of a technological advantage may include providing a femoral component and, in various embodiments, a patella implant, configured to provide a femoropatellar joint that allows for increased freedom-of-movement of the patella or patella implant, particularly in extension and/or partial states of flexion. Another non-limiting example of a technological advantage may include allowing increased freedom-of-movement that corresponds or more closely corresponds to the movement of the patella or patella implant in extension and/or partial states of flexion in native anatomy. A further non-limiting example of a technological advantage may include providing a knee implant having a reduced (or even eliminated) femoropatellar problems, particularly residual pain or discomfort in the anterior knee due to the kinematics of the femoropatellar joint.

FIG. 1 shows a perspective view of an embodiment of a left knee prosthesis. As shown in FIG. 1, a knee prosthesis 100 may be designed to replace at least a portion of a left knee joint between the distal end of a patient’s femur and the proximal end of a patient’s tibia. A mirror image (not shown) of knee prosthesis 100 may be configured to replace at least a portion of a right knee between the distal end of a patient’s femur and the proximal end of a patient’s tibia.

Knee prosthesis 100 may include a femoral component 200 for mounting to a distal end of a patient’s femur, a tibial component 300 for mounting to a proximal end of a patient’s tibia, and an insert 400 positioned between the femoral component 200 and the tibial component 300. Femoral component 200 may include a medial condylar section 202, a lateral condylar section 204 and a trochlear groove 206 joining anterior portions 214, 216 of medial and lateral condylar sections 202, 204 together.

Medial and lateral condylar sections 202, 204 may be disposed apart from one another to form an intercondylar recess or notch 208. Each condylar section 202, 204 may have an outer surface 210, 212 for contacting, engaging, etc. the tibial component 300 or the insert 400. Outer surfaces 210, 212 of each condylar section 202, 204 may have a distal portion (not shown) for contacting, engaging, etc. a portion of the tibial component 300 or the insert 400 when the knee joint is extended and partially flexed, and posterior portions 222, 224 for contacting, engaging, etc. a portion of the tibial component 300 or the insert 400 when the knee joint is flexed at angles of substantially 90 degrees or greater.

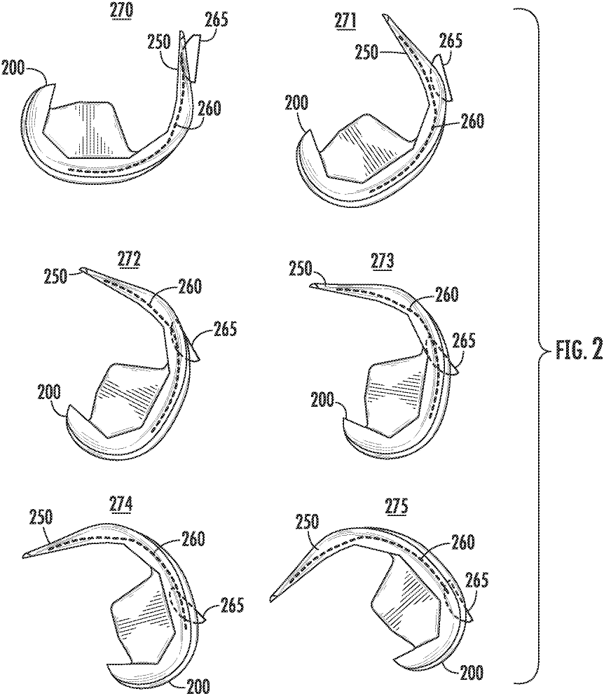

An anterior flange 250 may extend proximally from the medial and lateral condylar sections 202, 204 and intercondylar notch 208 to articulate with a patella or patella implant (not shown; see, for example, FIG. 2).

In some examples, the femoral component 200 may have a thickness approximately matching the bone resection necessary for the TKA. In various examples, the femoral component 200 may have a lateral condylar section 204 that is different in geometry than the geometry of the medial condylar section 202. For example, the anterior size of the lateral condylar section 204 may be larger than the anterior size of the medial condylar section 202. In some examples, the femoral component 200 may include a rounded medial profile. Examples are not limited in this context.

In the native human knee, the patella glides on the femoral condyles from full extension to full flexion. By about 20 degrees to about 30 degrees of flexion, the patella first begins to articulate within the trochlear groove 206. At extreme flexion, the patella lies in the intercondylar recess 208. Initially the patella contact occurs distally and with increased flexion, eventually the contact areas shift proximally on the patella. Patellofemoral contact force is substantially body weight when walking and increases to substantially 5 times body weight when stair climbing. These contact forces therefore impose a substantial load on the knee joint, which prostheses according to certain embodiments address to provide a knee implant with reduced (or even eliminated) femoropatellar joint pain and/or discomfort after the TKA procedure.

FIG. 2 shows a side view of a knee prosthesis showing the kinematics of a knee prosthesis (e.g., femoral component and patella) during extension and varying degrees of flexion. As shown in FIG. 2, a patella 265 may travel along a patella path 260 during (full) extension 270 and flexion 271-275 (about 30 degrees, about 60 degrees, about 90 degrees, about 120 degrees, and about 150 degrees flexion, respectively) along anterior flange 250. The dotted line of patella path 260 indicate the path of travel of patella 265 in contact with an anterior surface of femoral component 200 during extension 270 and various degrees of flexion 271-275.

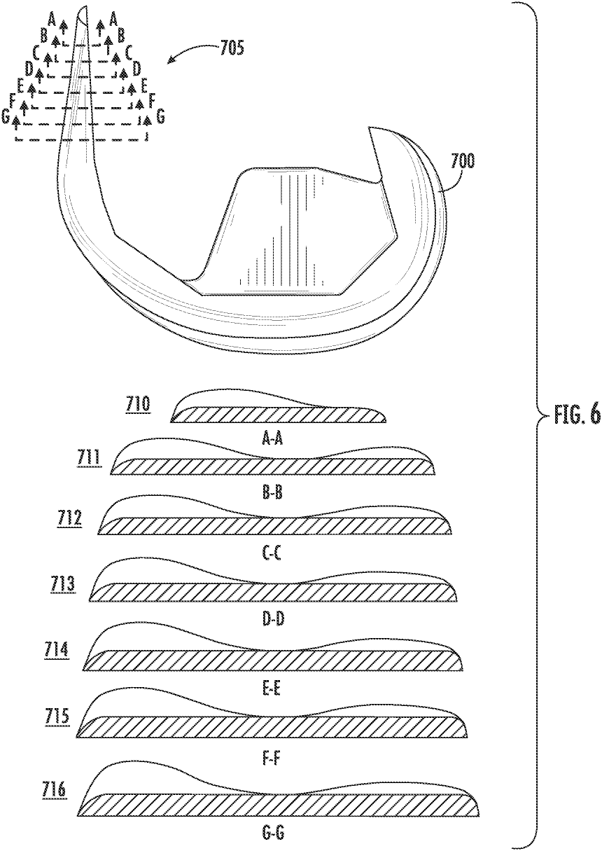

In some embodiments, patella 265 may be a natural human patella. In various embodiments, patella 265 may be a prosthetic patella. As such, reference to a patella herein is intended to include both a natural human patella and a patella implant unless explicitly stated otherwise. In some embodiments, a prosthetic patella may have various shapes known to those of skill in the art, including, without limitation round dome/sombrero, centralized oval, offset oval, anatomic, and mobile bearing. In some embodiments, as described in more detail in the present disclosure, patella 265 may include one or more raised portions, such as a raised distal portion (see, for example, FIG. 6).

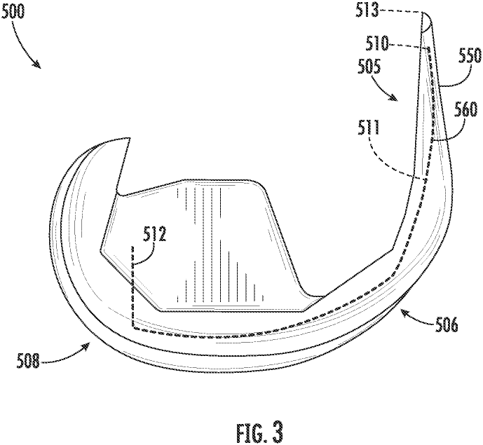

FIG. 3 shows a side view of an embodiment of a femoral component 500 that may be used in a knee prosthesis such as, for example, knee prothesis 100, in accordance with the present disclosure. As shown in FIG. 3, the femoral component 500 includes a patella path 560. In one embodiment, the patella path 560 extends along anterior flange 550 from a path starting region 510 (extension) to a path end region 512 at intercondylar notch 508 (flexion). A portion of the patella path 560 may include a reduced constraint region 505, for example, extending from a transition region 511 to starting region 510 (or, in some embodiments, to the top 513 of anterior flange 550). Another portion of the patella path 560 may include a constraint region, for example, in the form of trochlear groove 506 extending from end region 512 (i.e., intercondylar notch 508) to transition region 511. In general, transition region 511 may be a starting point, line, region, or other element associated with the inferior start of reduced constraint region 505 (i.e., the end closest to the trochlear groove 506).

In some embodiments, transition region 511 may provide a gradual transition between trochlear groove 506 and reduced constraint region 505. For example, trochlea conformity may gradually develop distal to reduced constraint region 505 starting inferior to transition region 511.

In various embodiments, a patella (for instance, patella 265 of FIG. 2) may be positioned (e.g., travel or move) within reduced constraint region 505 when the femoral component 500 is in extension (i.e., 0 degrees flexion) or partial flexion below a constrained flexion degree. In some embodiments, the constrained flexion degree may be less than about 1° flexion, about 1° flexion, about 2° flexion, about 3° flexion, about 4° flexion, about 5 degrees flexion, about 10 degrees flexion, about 15 degrees flexion, about 20 degrees flexion, about 30 degrees flexion, about 60 degrees flexion, and any value or range between any two of these values (including endpoints). Accordingly, in some embodiments, a patella may travel through reduced constraint region 505 and trochlear groove 506 during extension and flexion of the femoral component 500.

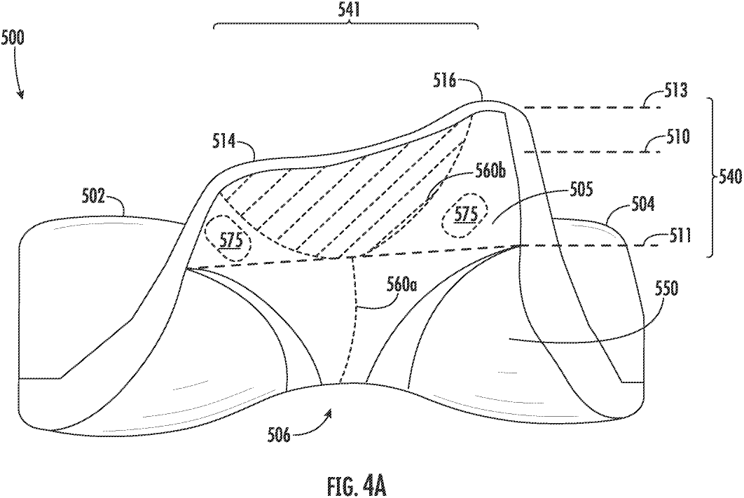

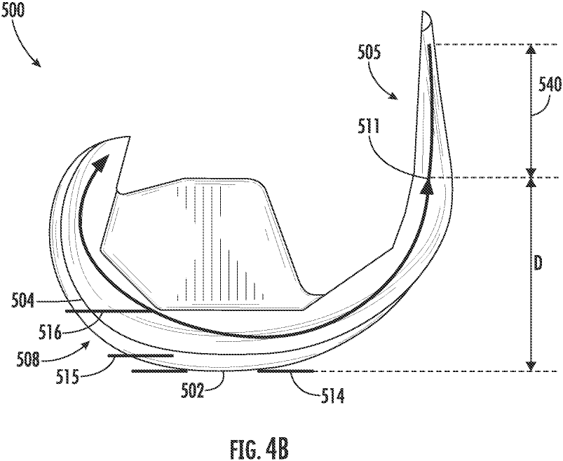

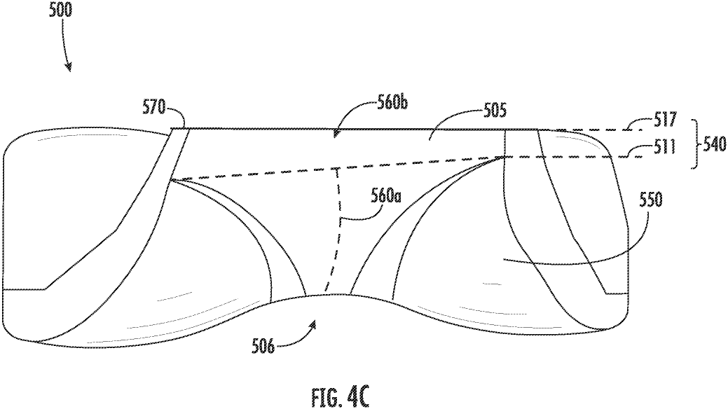

FIG. 4A shows a front view of an embodiment of a femoral component 500 that may be used in a knee prosthesis such as, for example, knee prosthesis 100, in accordance with the present disclosure. As shown in FIG. 4A, the femoral component 500 may include a reduced constraint region 505 formed on a portion of the femoral component 500. In some embodiments, the location, size, and/or other characteristics may be based in relation to various portions of the femoral component 500, such as anterior flange 550, medial anterior portion 514, lateral anterior portion 516, articular surface and/or portions thereof, medial condylar section 502, lateral condylar section 504, (distal) knee resection area or boundary, and/or the like.

In some embodiments, reduced constraint region 505 may be formed in a superior portion of the anterior flange 550. In some embodiments, reduced constraint region 505 may have a length 540, for example, extending from top or superior region 513 of the anterior flange 550 (or starting region 510) to transition region 511. In general, the length 540 of the reduced constraint region 505 may be determined to provide the femoral component 500 with a trochlear groove 506 that extends less superior from the distal femoral condyles 502, 504, as compared with a conventional knee implant, to allow more freedom-of-movement of the patella within the reduced constraint region 505.

In various embodiments, the length 540 of the reduced constraint region 505 may be determined based on a percentage of the anterior-posterior length of the femoral component 500 or the anterior flange 550 (i.e., a percentage of the length of the distal portion of the femoral component 500 or anterior flange 550 that is covered by reduced constraint region 505, for instance at a distal portion of the femoral component 500 or the anterior flange 550) at an end opposite the intercondylar notch 508). For example, the length 540 of the reduced constraint region 505 may be a length percentage of the femoral component 500 of less than about 1 percent, less than about 5 percent, less than about 10 percent, less than about 15 percent, less than about 20 percent, less than about 30 percent, less than about 40 percent, less than about 50 percent, less than about 60 percent, less than about 70 percent, less than about 80 percent, less than about 90 percent, less than about 100 percent, about 1 percent, about 5 percent, about 10 percent, about 15 percent, about 20 percent, about 30 percent, about 40 percent, about 50 percent, about 60 percent, about 70 percent, about 80 percent, about 90 percent, about 95 percent, and any value or range between any two of these values (including endpoints). In another example, the length 540 of the reduced constraint region 505 may be a length percentage of the anterior flange 550 of less than about 1 percent, less than about 5 percent, less than about 10 percent, less than about 15 percent, less than about 20 percent, less than about 30 percent, less than about 40 percent, less than about 50 percent, less than about 60 percent, less than about 70 percent, less than about 80 percent, less than about 90 percent, less than about 100 percent, about 1 percent, about 5 percent, about 10 percent, about 15 percent, about 20 percent, about 30 percent, about 40 percent, about 50 percent, about 60 percent, about 70 percent, about 80 percent, about 90 percent, about 95 percent, and any value or range between any two of these values (including endpoints).

In some embodiments, the length 540 of the reduced constraint region 505 may be a discrete value. For example, the length 540 of the reduced constraint region 505 may be less than about 0.25 mm, less than about 0.5 mm, less than about 1 mm, less than about 2 mm, less than about 3 mm, less than about 4 mm, less than about 5 mm, less than about 6 mm, less than about 7 mm, less than about 8 mm, less than about 9 mm, less than about 10 mm, less than about 11 mm, less than about 12 mm, less than about 13 mm, less than about 14 mm, less than about 15 mm, less than about 16 mm, less than about 17 mm, less than about 18 mm, less than about 19 mm, less than about 20 mm, less than about 21 mm, less than about 22 mm, less than about 23 mm, less than about 24 mm, less than about 25 mm, about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, and any value or range between any two of these values (including endpoints).

In some embodiments, the length 540 of the reduced constraint region 505 may be or may be determined to ensure that the patella remains within the reduced constraint region 505 during a threshold percentage of flexion (with extension being about 0 degrees flexion). For example, the threshold percentage of flexion may be about 0 degrees flexion, less than about 1 degree flexion, less than about 5 degrees flexion, less than about 10 degrees flexion, less than about 15 degrees flexion, less than about 20 degrees flexion, less than about 25 degrees flexion, less than about 30 degrees flexion, less than about 35 degrees flexion, less than about 40 degrees flexion, less than about 45 degrees flexion, less than about 50 degrees flexion, less than about 55 degrees flexion, less than about 60 degrees flexion, about 1° flexion, about 5 degrees flexion, about 10 degrees flexion, about 15 degrees flexion, about 20 degrees flexion, about 25 degrees flexion, about 30 degrees flexion, about 35 degrees flexion, about 40 degrees flexion, about 45 degrees flexion, about 50 degrees flexion, about 55 degrees flexion, about 60 degrees flexion, and any value or range between any two of these values (including endpoints).

In various embodiments, a width 541 of the reduced constraint region 505 may be determined based on a percentage of the anterior-posterior width of the femoral component 500 (i.e., a percentage of the width of the femoral component 500 or the anterior flange 550 that is covered by the reduced constraint region 505). For example, the width 541 of the reduced constraint region 505 may be a width percentage of the femoral component 500 of less than about 1 percent, less than about 5 percent, less than about 10 percent, less than about 15 percent, less than about 20 percent, less than about 30 percent, less than about 40 percent, less than about 50 percent, less than about 60 percent, less than about 70 percent, less than about 80 percent, less than about 90 percent, less than about 100 percent, about 1 percent, about 5 percent, about 10 percent, about 15 percent, about 20 percent, about 30 percent, about 40 percent, about 50 percent, about 60 percent, about 70 percent, about 80 percent, about 90 percent, about 95 percent, about 100 percent, and any value or range between any two of these values (including endpoints).

In some embodiments, the width 541 of the reduced constraint area 505 may be a discrete value (for example, spanning equally across a midline of the femoral component 500). For example, the width 541 of the reduced constraint region 505 may be less than about 0.25 mm, less than about 0.5 mm, less than about 1 mm, less than about 2 mm, less than about 3 mm, less than about 4 mm, less than about 5 mm, less than about 6 mm, less than about 7 mm, less than about 8 mm, less than about 9 mm, less than about 10 mm, less than about 11 mm, less than about 12 mm, less than about 13 mm, less than about 14 mm, less than about 15 mm, less than about 16 mm, less than about 17 mm, less than about 18 mm, less than about 19 mm, less than about 20 mm, less than about 21 mm, less than about 22 mm, less than about 23 mm, less than about 24 mm, less than about 25 mm, about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, and any value or range between any two of these values (including endpoints).

Accordingly, as shown in FIG. 4A, some embodiments may provide a knee implant with a femoral component 500 having a reduced constraint (i.e., flattened) region 505 in a proximal anterior flange 550 region where a conforming trochlea groove would typically be. In various embodiments, trochlea conformity of trochlea groove 506 may develop gradually distal to the reduced constraint region 505, below transition region 511.

FIG. 4B shows a side view of an alternate embodiment of a femoral component 500 that may be used in a knee prosthesis such as, for example, knee prosthesis 100, in accordance with the present disclosure. As illustrated, in some embodiments, the transition region 511 (i.e., the start of the reduced constraint region 505) may be based on the distal medial articular surface 502 or the distal lateral articular surface 504. For example, the transition region 511 may be a distance (i.e., distance D) from an end 514 of the medial articular surface 502 or an end 515 of the distal lateral articular surface 504. In various embodiments, the transition region 511 may be based on a knee resection region 516. For example, in some embodiments, the transition region 511 may be a discrete distance superior to end 514, end 515, and/or region 516, including, without limitation, about 5 mm, about 10 mm, 15 mm, about 20 mm, about 25 mm, about 30 mm, about 35 mm, about 40 mm, about 45 mm, about 50 mm, about 60 mm, about 70 mm, about 80 mm, and any value or range between any two of these values (including endpoints).

FIG. 4C shows a front view of an alternate embodiment of a femoral component 500 that may be used in a knee prosthesis such as, for example, knee prothesis 100, in accordance with the present disclosure. In some embodiments, the femoral component 500 includes an anterior tip 570 that is truncated, for example, at or just above the transition region 511. In such an embodiment, the reduced constraint region 505 may extend from the transition region 511 to the end region 517. In some embodiments, the length 540 of the reduced constraint region 505 in the embodiment of FIG. 4C may be about 1 mm, about 5 mm, about 10 mm, about 20 mm, and/or any value or range between any two of these values (including endpoints).

In various embodiments, referring to FIGS. 4A and 4C, the trochlea groove 506 and the reduced constraint region 505 may operate to provide patella path portions 560a, 560b with different degrees of freedom-of-movement of a patella. For example, a patella may have increased medial-lateral movement within path portion 560b located in reduced constraint region 505 as compared with path portion 560a located in trochlear groove 506. In some embodiments, reduced constraint region 505 may be configured to provide a percentage medial-lateral movement increase within reduced constraint region 505 as compared with trochlear groove 506. For instance, a patella may have a constrained medial-lateral movement value or range (e.g., 1 mm) within trochlear groove 506 and an increased medial-lateral movement value or range within the reduced constraint region 505 (e.g., the increased medial-lateral movement value or range within the reduced constraint region 505 may be a percentage increase over the constrained medial-lateral movement value or range within the trochlear groove 506). In some embodiments, the percentage increase of medial-lateral movement within the reduced constraint region 505 may be about 105 percent, about 110 percent, about 125 percent, about 150 percent, about 300 percent, about 300 percent, about 400 percent, about 500 percent, about 1000 percent, greater than about 1000 percent, and any value or range between any two of these values (including endpoints). In some embodiments, the reduced constraint region 505 may be configured to provide a discrete value of medial-lateral movement (or movement increase compared with a conventional device) within the reduced constraint region 505 as compared with the trochlear groove 506, for instance, of about 0.25 mm, about 0.5 mm, about 1 mm, about 5 mm, about 10 mm, 20 mm, greater than about 20 mm, and any value or range between any two of these values (including endpoints).

In another example, a patella may have increased internal-external rotation within the path portion 560b in the reduced constraint region 505 compared with the internal-external rotation provided in the path portion 560a in the trochlear groove 506. For instance, a patella may have a constrained internal-external rotation value or range within the trochlear groove 506 and an increased internal-external rotation value or range within the reduced constraint region 505 (e.g., the increased internal-external rotation value or range within the reduced constraint region 505 may be a percentage increase over the constrained internal-external rotation value or range within the trochlear groove 506). In some embodiments, the percentage increase of internal-external rotation within the reduced constraint region 505 may be about 105 percent, about 110 percent, about 125 percent, about 150 percent, about 300 percent, about 300 percent, about 400 percent, about 500 percent, about 1000 percent, greater than about 1000 percent, and any value or range between any two of these values (including endpoints). In some embodiments, the reduced constraint region 505 may be configured to provide a discrete value of internal-external rotation increase within the reduced constraint region 505 as compared with the trochlear groove 506, for instance, of about 1°, about 5 degrees, about 10 degrees, about 20 degrees, about 30 degrees, and any value or range between any two of these values (including endpoints).

In various embodiments, one or more stops 575 (FIG. 4A) may be arranged within the reduced constraint region 505. In general, the stops 575 may be positioned to provide a limit or border for patella freedom-of-movement, such as medial-lateral movement. In some embodiments, the stops 575 may be or may include, without limitation, projections, bumps, ridges, and/or the like.

In alternative embodiments, the anterior flange 570 of the femoral component 500 may be truncated, for example, where at the superior starting location of the reduced constraint region 505 and/or reducing conformity (e.g., groove depth, sulcus angle) of the reduced constraint region 505 instead of “flattening” reduced constraint region 505.

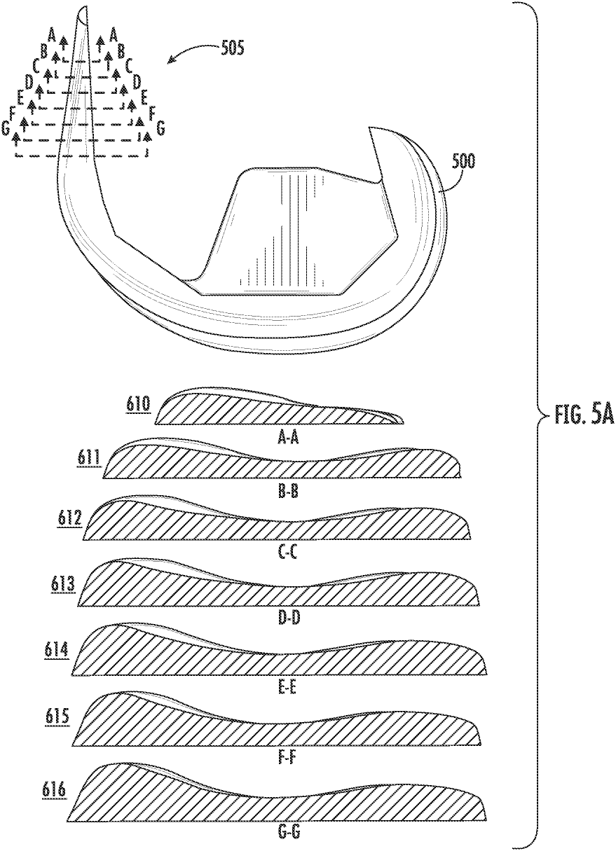

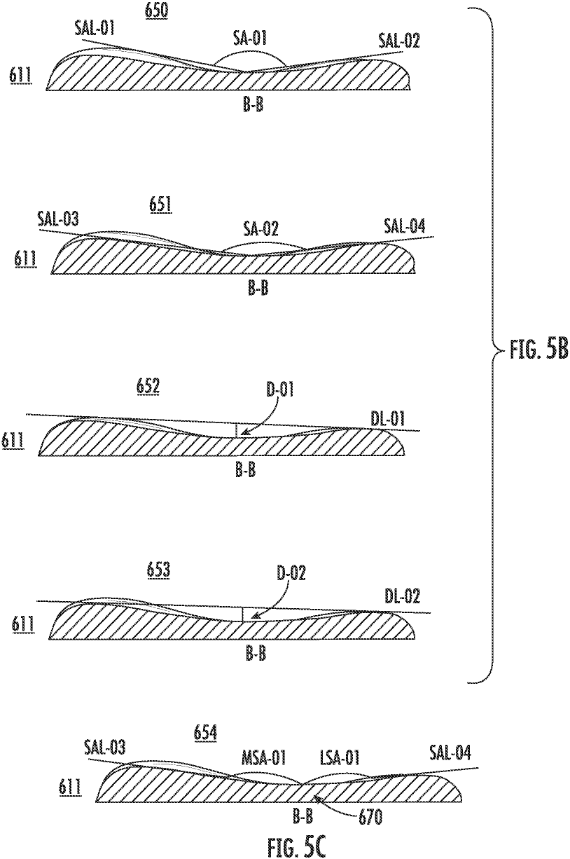

FIG. 5A shows a side view and corresponding sectional views of an embodiment of a femoral component 500 that may be used in a knee prosthesis such as, for example, knee prosthesis 100, in accordance with the present disclosure. As shown in FIG. 5A, transverse sectional views 610-616 through reduced constraint region 505 illustrate the contrast between an exemplary embodiment of a femoral component in accordance with features of the present disclosure and an exemplary conventional device, for example, having a trochlear groove or other constraint region extending to the top of the anterior flange. As illustrated, a femoral component 500 including a reduced constraint region in accordance with one or more features of the present disclosure is shown with shading. An exemplary conventional implant is shown without shading. As indicated in FIG. 5A, the reduced constraint region 505 may be flatter than the same region of the exemplary conventional device.

FIG. 5B shows patella track information for an illustrative sectional view of FIG. 5A in accordance with the present disclosure. The reduced constraint or “flatness” of reduced constraint region 505 may correspond with various constraint factors, such as a sulcus angle, groove depth, and/or the like. View 650 depicts a sulcus angle measurement for the exemplary conventional device of section 611 of FIG. 5A. In general, sulcus angle SA-01 may be measured using sulcus angle lines SAL-01 and SAL-02 crossing through both condylar eminences. View 651 depicts sulcus angle SA-02 for a femoral component 500 in accordance with one or more features of the present disclosure of section 611 of FIG. 5A. As shown in FIG. 5B, SA-02 > SA-01 for the same region of femoral component. In general, a sulcus angle for a trochlear groove of an exemplary conventional device may be less than about 160 degrees, less than about 150 degrees, or, more typically, less than about 145 degrees. In comparison, the reduced constraint region 505 may have areas having a sulcus angle of greater than about 145 degrees, greater than about 150 degrees, greater than about 160 degrees, greater than about 170 degrees, equal to or greater than about 180 degrees, or any value or range between any two of these values (including endpoints). In some embodiments, sulcus angles within reduced constraint region 505 may be greater than about 150 degrees. In various embodiments, sulcus angles within reduced constraint region 505 may be greater than about 160 degrees. The sulcus angle or angles within reduced constraint region 505 may be a maximum value or an average value.

View 652 depicts a groove depth measurement for the exemplary conventional device of section 611 of FIG. 5A. In general, groove depth may be measured as the distance between a line drawn tangential to the anterior margin of the medial and lateral femoral condyles and the deepest part of the groove. Groove depth D-01 may be measured based on tangential depth line DL-01 for the exemplary conventional device. View 652 depicts groove depth D-02 based on tangential line DL-02 for the same region of a device configured in accordance with one or more features of the present disclosure. As shown in FIG. 5B, D-02 < D-01. In some embodiments, the groove depth of areas of reduced constraint region 505 may be less than about 4 mm, less than about 3 mm, less than about 2 mm, less than about 1 mm, less than about 0.5 mm, less than about 0.25 mm, about 0 mm, or any value or range between any two of these values (including endpoints).

FIG. 5C shows patella track information for an illustrative sectional view of FIG. 5A in accordance with the present disclosure. View 654 depicts a sulcus angle measurement for the exemplary conventional device of section 611 of FIG. 5A. As shown in FIG. 5C, a plurality of sulcus angles may be determined based on various anatomical points of reference. For example, a lateral sulcus angle LSA-01 or a medial sulcus angle MSA-01 may be determined relative to a medial-lateral axis 670. In some embodiments, medial-lateral axis 670 may be defined relative to Whiteside’s line, an anterior-posterior axis, an epicondylar axis, a posterior condylar axis, and/or the like. Lateral sulcus angle LSA-01 may be an angle between SAL-03 (or other sulcus angle reference line) and medial-lateral axis 670 and/or medial sulcus angle MSA-01 may be an angle between SAL-04 (or other sulcus angle reference line) and medial-lateral axis 670.

FIG. 6 shows a side view and corresponding sectional views of an embodiment of a femoral component 700 that may be used in a knee prosthesis such as, for example, knee prosthesis 100, in accordance with the present disclosure. In accordance with one or more features of femoral component 700, the femoral component 700 includes a patella path having a reduced constraint region 705. In accordance with the features of the femoral component 700, the reduced constraint region 705 may be provided as a completely flat surface on the anterior flange.

As shown in FIG. 6, transverse sectional views 710-716 through reduced constraint region 705 illustrate the contrast between an exemplary embodiment of a femoral component having a completely flat surface on the anterior flange in accordance with features of the present disclosure and an exemplary conventional device, for example, having a trochlear groove or other constraint region extending to the top of the anterior flange. As illustrated, a femoral component 700 including a reduced constraint region in accordance with one or more features of the present disclosure is shown with shading. An exemplary conventional implant is shown without shading. As indicated in FIG. 6, the reduced constraint region 705 is completely flat and thus flatter than the same region of the exemplary conventional device.

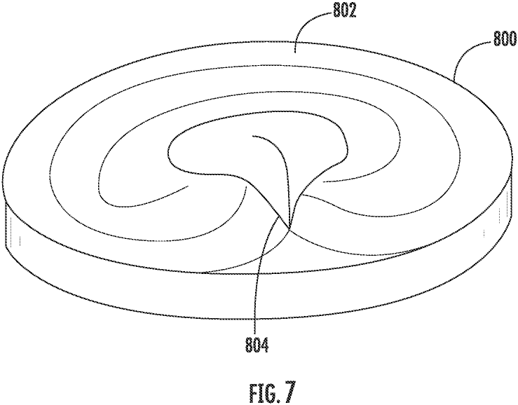

FIG. 7 shows a perspective view of an embodiment of a patella implant 800 in accordance with the present disclosure. In extension (and certain states of partial flexion), the patella implant 800 may be located in the reduced constraint region at various potential medial-lateral positions. As the knee starts to flex, the trochlear groove of the femoral component and the patella implant 800 would need to guide the patella implant 800 into the trochlear groove from the wide range of potential medial-lateral positions. Accordingly, in some embodiments, the distal portion of the posterior side of the patella implant 800, which is more likely a functional contact area in extension than flexion, may be formed to aid the transition of the patella implant 800 into the trochlear groove (or other constraint region) during early flexion.

As shown in FIG. 7, a patella implant 800 according to some embodiments may have a raised or transitioning portion 804 arranged on a distal region of the posterior side 802 of patella implant 800. During flexion, as the patella implant 800 moves from the reduced constraint region to the trochlear groove (or other constraint region), the raised portion 804 of the patella implant 800 may operate to guide the patella implant 800 into the trochlear groove. As shown in FIG. 7, the raised portion 804 may be formed to flair out moving from the outer edge to the center of posterior side 802.

The patella implant 800 may be formed of various materials known to those of skill in the art, including metals, ceramics, composites, fiber reinforced composites, polymers, Ultra Highly Cross-Linked Polyethylene (UHXLPE), Ultra High Molecular Weight PolyEthylene (UHMWPE), combinations thereof, and/or the like.

In some embodiments, the raised portion 804 may have a height (for example, based on a difference between the lowest point of the posterior side 802 and the highest point of the raised portion 804) of greater than or equal to about 0.1 mm, greater than or equal to about 0.2 mm, greater than or equal to about 0.3 mm, greater than or equal to about 0.4 mm, greater than or equal to about 0.5 mm, greater than or equal to about 1 mm, greater than or equal to about 2 mm, greater than or equal to about 3 mm, greater than or equal to about 4 mm, greater than or equal to about 5 mm, and any values or ranges between any two of these values (including endpoints). In some embodiments, the raised portion 804 may have a length of greater than or equal to about 0.1 mm, greater than or equal to about 0.2 mm, greater than or equal to about 0.3 mm, greater than or equal to about 0.4 mm, greater than or equal to about 0.5 mm, greater than or equal to about 1 mm, greater than or equal to about 2 mm, greater than or equal to about 3 mm, greater than or equal to about 4 mm, greater than or equal to about 5 mm, and any values or ranges between any two of these values (including endpoints). In some embodiments, the location of the raised portion 804 may be determined based on various factors, such as whether the patella implant 800 tracks medial, lateral, or central in extension. In various embodiments, the sulcus angle and/or groove depth of a reduced constraint region may be determined based on the height of raised portion.

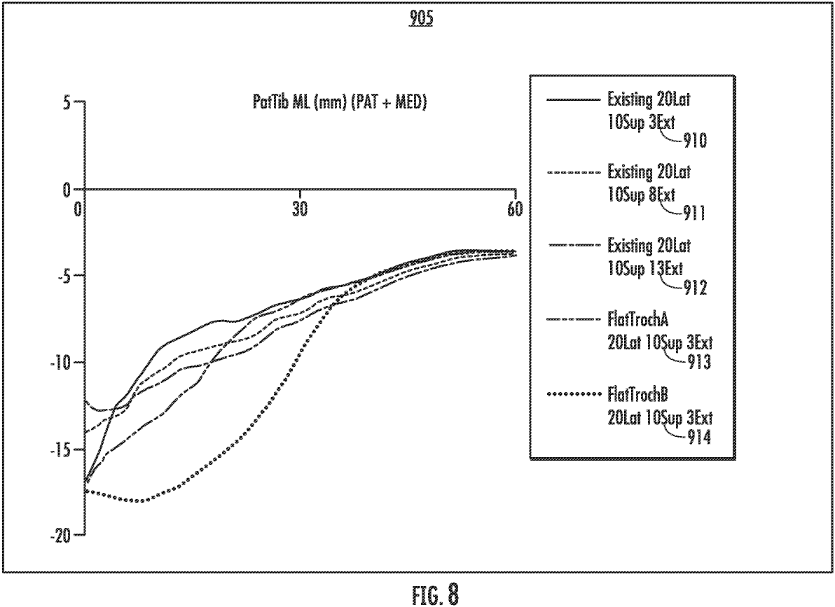

FIG. 8 shows a graph of patella medial-lateral tracking during degrees of knee flexion for simulated knee prosthesis in accordance with the present disclosure. Graph 905 depicts knee implant simulation data generated via LifeMOD™/KneeSIM provided by LifeModeler, Inc. of San Clemente, California, United States. More specifically, graph 905 depicts knee implant simulation data of the patella medial-lateral tracking relative to the tibia for a condition where the initial position of the patella is more lateral and superior than a typical patient comparing an existing implant positioned at a typical 3° external rotation (910) compared to 8° external rotation (911), and 13° external rotation (912) where increased external rotation smooths the lateral translation of the patella as the knee extends. Graph 913 illustrates an implant modified according to one or more features of the present disclosure, the modified implant including a reduced constraint region (i.e., a flattened trochlea). As illustrated, the reduced constraint region smooths the lateral translation of the patella as the knee extends and, in addition, without the downside of affecting the flexion balance of the knee. Graph 914 illustrates an implant modified to include a reduced constraint region wherein the reduced constraint region includes a completely flattened surface, which provides increased patella freedom.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments or configurations.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader’s understanding of the present disclosure, and do not create limitations, particularly as to the position, orientation, or use of this disclosure.

Connection references (e.g., engaged, attached, coupled, connected, affixed, mounted, joined, and/or the like) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A knee implant comprising:
a femoral component arranged and configured to engage a patient’s femur, the femoral component including an articular surface arranged and configured to articulate relative to a tibial component or insert;
wherein the articular surface includes a patella path along which a patella travels, the patella path including a constraint region and a reduced constraint region, the reduced constraint region being arranged and configured to provide increased freedom-of-movement of the patella as it travels over the reduced constraint region compared to freedom-of-movement of the patella as it travels over the constraint region.

2. The knee implant of claim 1, wherein the increased freedom-of-movement of the patella provided by the reduced constraint region enables increased medial-lateral movement of the patella.

3. The knee implant of claim 2, wherein the increased medial-lateral movement of the patella within the reduced constraint region of the patella path, as compared with medial-lateral movement of the patella within the constraint region of the patella path, is about 105 percent to about 150 percent greater.

4. The knee implant of claim 1, wherein the increased medial-lateral movement of the patella within the reduced constraint region of the patella path, as compared with medial-lateral movement of the patella within the constraint region of the patella path, is about 1 mm to about 20 mm greater.

5. The knee implant of claim 1, wherein the increased freedom-of-movement of the patella provided by the reduced constraint region enables increased internal-external rotation of the patella.

6. The knee implant of claim 5, wherein the increased internal-external rotation of the patella within the reduced constraint region of the patella path, as compared with internal-external rotation of the patella within the constraint region of the patella path, is about 105 percent to about 150 percent greater.

7. The knee implant of claim 1, wherein the constraint region of the patella path comprises a trochlear groove positioned inferiorly to the reduced constraint region.

8. The knee implant of claim 7, wherein the patella travels within the reduced constraint region during extension and within the trochlear groove during flexion.

9. The knee implant of claim 8, wherein the femoral component is arranged and configured so that the patella resides within the reduced constraint region when the patella travels less than about 10 degrees of flexion.

10. The knee implant of claim 1, wherein the reduced constraint region includes a flattened region formed adjacent to a proximal anterior flange of the femoral component.

11. The knee implant of claim 1, wherein the reduced constraint region has a length of about 1 percent to about 20 percent of a length of a distal anterior flange region.

12. The knee implant of claim 1, wherein the reduced constraint region has a length of 1 mm to about 20 mm.

13. The knee implant of claim 1, further comprising a patella implant arranged and configured to travel along the patella path formed in the articular surface of the femoral component, wherein the patella implant includes a raised portion arranged and configured on a distal region of a posterior side of the patella implant.

14. The knee implant of claim 13, wherein the raised portion is arranged and configured to facilitate engagement of the patella implant within the constraint region of the patella path.

15. The knee implant of claim 14, wherein the raised portion is arranged and configured to flair out moving from an outer edge to a center of posterior side.
